(19) United States
(12) Patent Application Publication
(10) Pub. No.: US 2023/0341364 A1
(43) Pub. Date: Oct. 26, 2023

(54) METHOD FOR DETERMINING ANTIOXIDANT CAPACITY IN BEVERAGES

(71) Applicant: ECOSPEC NOVELTECH PTE LTD, Singapore (SG)

(72) Inventor: Hwee Hong CHEW, Singapore (SG)

(21) Appl. No.: 18/090,679

(22) Filed: Dec. 29, 2022

Related U.S. Application Data
(60) Provisional application No. 63/333,663, filed on Apr. 22, 2022.

(51) Int. Cl. G01N 31/22; G01N 31/00

(52) U.S. Cl. CPC G01N 31/221; G01N 31/005

(57) ABSTRACT

The invention provides a method for determining the antioxidant capacity of beverage by titrating a standard oxidant solution using liquid sample of the beverage and monitoring oxidation reduction potential of the solution until a neutral reference value is reached, and using the titration volume to calculate the antioxidant capacity. The invention also provides test apparatus and test kit for performing the method of the invention.

### RELATED APPLICATION

This non-provisional application claims priority from provisional application No. 63/333,663 filed on Apr. 22, 2022, the disclosures of which are incorporated herein by reference in their entirety.

### FIELD OF THE INVENTION

This invention relates generally to a method for determining the antioxidant capacity in a liquid sample and test procedures thereof. Particularly, the present invention utilizes Oxidation Reduction (REDOX) Neutralization as the basis for the determination of antioxidant value of beverages.

### BACKGROUND OF THE INVENTION

Nowadays people are more and more concerned about healthy diet. Human body produces free radicals and Reactive Oxygen Species (ROS) in the metabolism process. Such free radicals and ROS may also be ingested into human body from environment. These free radicals and ROS are harmful to human body as they can kill healthy cells, damage cell membrane or DNA etc. causing many health issues. In the meantime, it is also known of certain antioxidant substances exist in food and beverages, which are beneficial to human body as they can neutralize the oxidizing free radicals and ROS.

Free radicals/ROS are damaging to human body because they are short of electron(s); they snatch electron from other healthy cells just to keep themselves stable but that kills other healthy cells. The purpose of consuming antioxidant substances is to donate free electron to these free radicals/ROS to minimize attack on healthy cells. For supplying antioxidants to human body, it is typically convenient that beneficial antioxidant contents are contained in the beverages we consumed every day. It is therefore important to know the quantity of free electron donating antioxidant content in the beverages that is beneficial to human body.

There are several methods adopted in the Food, Beverage and Nutrition industries to determine the free radicals/ROSs neutralizing antioxidant content in beverages or foods. Such evaluations include the ORAC (Oxygen Radical Absorbance Capacity) value, or direct measurement of certain kinds of antioxidant contents such as ascorbic acid content etc. The antioxidant content is then expressed in various ways such as using the Trolox equivalent in ORAC method, or using concentration of ascorbic acid in mg/L.

For example, oxygen radical absorbance capacity (ORAC) assay determines the quantity of antioxidant contained in solid foods and beverages using AAPH (2,2′-azobis(2-methylpropionamidine) dihydrochloride) as an ROS agent (i.e. standard oxidant) and Trolox® (6-hydroxy-2,5,7,8-tetramethylchromane-2-carboxylic acid) as a standard antioxidant for comparison. A Roche COBAS FARA II fluorescence analyser is used to measure relative fluorescent intensity (RFI) versus time of samples and Trolox references after AAPH was added. The summed RFI data were plotted versus different known concentrations of the sample and Trolox to generate regression curves. The Trolox Equivalent (TE) value of antioxidants in the sample is then derived by dividing the slope of the regression curve of the sample by the slope of the regression curve of Trolox. This Trolox equivalent value can be used as the ORAC value indicating antioxidant capability of the sample.

All prior art methods involve using complex equipment such as ORAC assay test equipment, complex test methods and procedures. For other antioxidant concentration information, unless the types of antioxidants are identified, it is difficult to determine and test the concentrations of all antioxidant contents present in the beverage. When the test methods and procedures are complex, expensive analytical instruments and well-trained lab chemists are required, which becomes difficult to implement across the industries or for the industries to adopt it as standard. There are other simpler methods such as using the Oxidation Reduction Potential (ORP) meter to check the ORP of the beverages. However, the ORP readings in milli Volt (mV) show only the oxidative or reductive potential and most commonly with reference to standard Ag/AgCl cell; it is unable to show the quantity of the oxidant or antioxidant content.

In view of the above as well as the increasing awareness of the beneficial antioxidant content in the beverages nowadays, it is necessary to have a low cost, easy to use method for determination, yet producing a good representative measurement of the total antioxidant capacity of the beverages which are health beneficial. With this measurement method, all the industries producing beneficial antioxidizing beverages will be able to determine the antioxidants in the beverages they produced, while it is also easier for quality control and regulators to verify the claimed antioxidant content in the beverages. This also benefits the consumers by providing them quantitative guideline for the actual health beneficial antioxidant content in beverages they are consuming.

### SUMMARY OF THE INVENTION

The present invention is developed to solve the problems noted above by providing an efficient and convenient method for determining antioxidant capacity of liquid beverages. For this purpose, the invention provides a new concept of Total Antioxidant Potential & Energy Capacity (TAPEC) for characterizing the antioxidizing content in liquid sample, which makes the antioxidant effect between different beverages being easily comparable, thus the ingested antioxidant contents can be easily monitored for a healthy diet.

In one aspect of the invention, provided is a method for determining the antioxidant capacity (TAPEC) of a liquid sample, comprising the steps of:
preparing the liquid sample by removing all undissolved contents therein;
measuring pH of the liquid sample and adjusting pH of the liquid sample to about 7 if the measured pH is higher than 7;
measuring oxidation reduction potential (ORP) of a standard oxidant;
determining a sample titration volume of the liquid sample necessary for bringing ORP of the standard oxidant of a pre-determined volume to a neutral reference value; and
calculating the antioxidant capacity based on the sample titration volume of the liquid sample.

In one embodiment of the invention, the standard oxidant is a solution of sodium hypochlorite. Preferably, the standard oxidant has a total residual oxidant (TRO) value of 0.2 ppm, and/or an ORP reading of +550 mV vs Ag/AgCl reference cell.

In certain embodiment of the invention, the method further comprising the step of:
preparing the standard oxidant by adding at a ratio of 8 μL of a 10% sodium hypochlorite solution into 1 L of pure water.

In one preferred embodiment of the invention, the method further comprising the step of:
preparing a standard antioxidant, wherein the standard antioxidant is a solution of ascorbic acid.

In certain embodiment of the invention, the standard antioxidant comprises ascorbic acid at a concentration of 800 mg/100 mL, and/or has an ORP reading of +175 mV vs Ag/AgCl reference cell. For instance, the standard antioxidant is prepared by adding at a ratio of 800 mg of pure ascorbic acid into 100 mL of pure water.

In another preferred embodiment of the invention, the method further comprising the steps of:
determining a reference titration volume of the standard antioxidant necessary for bringing ORP of the standard oxidant of a pre-determined volume to a neutral reference value; and
calculating the antioxidant capacity based on the sample titration volume and the reference titration volume.

In one specific embodiment of the invention, the antioxidant capacity is calculated according to the following formula:

$$antioxidant scaler value ( asv ) = \frac{refe r ence titration volume \times 1000}{sample titration volume} .$$

In certain embodiment of the invention, the neutral reference value used in the method is an ORP reading of +375 mV vs Ag/AgCl reference cell.

In a yet preferred embodiment of the invention, the method further comprising the steps of:
preparing pure water;
using the pure water as a neutral liquid having ORP of the neutral reference value; and
using the pure water for the preparation of standard oxidant and standard antioxidant.

In a specific embodiment of the invention, pH of the liquid sample with pH higher than 7 is adjusted by adding hydrochloric acid solution.

In certain embodiment of the invention, the method further comprising the step of:
calibrating pH meter and ORP meter before measuring pH and ORP, respectively.

In another aspect of the invention, provided is a test apparatus for performing the TAPEC measurement of the invention, comprising one or more of the following:
a pH meter for measuring pH of a liquid sample;
an ORP meter for measuring ORP of a standard oxidant; and
a titration device for determining titration volume of the liquid sample.

In yet another aspect of the invention, provided is a test kit for performing the TAPEC measurement of the invention, comprising one or more of the following:
test apparatus for performing the TAPEC measurement;
pure water;
hydrochloric acid; and
standard chemicals of the standard oxidant and the standard antioxidant, such as sodium hypochlorite and ascorbic acid, respectively.

### DETAILED DESCRIPTION OF THE INVENTION

This invention will be illustrated and described by way of preferred embodiments. It is understood that the invention may be performed in many different configurations and forms with different materials and devices.

### Total Antioxidant Potential & Energy Capacity (TAPEC)

In present invention, TAPEC value is determined using Oxidation Reduction (REDOX) Neutralization as the basis for calculation. ORP meter is used in TAPEC method of the invention to monitor the neutralization progress. In order to determine total capacity of free radicals/ROS neutralization, the TAPEC method utilizes a pre-determined neutral reference set point in ORP reading indicating complete neutralization of a test sample.

Most ORP meters available in the market used Ag/AgCl as reference cell, although other reference cells such as calomel cell etc. can also be used in the lab. For an ORP meter using Ag/AgCl reference cell, the measurement value of zero mV means the ORP of the measured liquid has the same redox potential as Ag/AgCl cell. However, for neutral TAPEC reference set point of the present invention, ORP mV potential of neutral water which contains no oxidant nor antioxidant is used as the reference to determine whether the test sample is completely neutralized to non-oxidizing and non-antioxidizing state.

In all redox reactions, oxidant and antioxidant are both present. When the oxidant is completely neutralized by the antioxidant, and at the same time the antioxidant is completely neutralized by the oxidant, the ORP of the neutralized solution or liquid is defined as the true neutralized solution ORP. Pure water having electrical conductivity of few micro-Siemens (has/cm) contains virtually no oxidant or antioxidant. The ORP of this pure water is therefore used and defined as the ORP of a neutral solution. When using Ag/AgCl referenced ORP meter for measurement, the ORP of the neutral pure water gives a reading of +375 mV. This +375 mV is therefore used as TAPEC neutralization titration end point or neutral reference set point.

Specifically, there are two reasons for choosing pure water as the reference potential:
Neutral pure water does not contain oxidant or antioxidant hence a good representation for complete neutralization point.
Water is most relevant to cell metabolism in human body. Water is the end product of the oxidative phosphorylation process of the cell metabolism after electron transport chain.

4H⁺+O₂+4e⁻→H₂O

In present invention, TAPEC neutralization end set point ORP reading is defined as +375 mV (when using Ag/AgCl referenced ORP meter). Said ORP reading is used to determine whether the TAPEC neutralization test has attained its complete neutralization status.

### Standard Oxidant

In TAPEC calculation, a standard oxidant solution is used as reference oxidizing agent to neutralize different test beverages. For stability, reliability, repeatability and ease of use, a pre-determined amount of oxidant is added to one litre of pure water to produce a pre-determined amount of Total Residual Oxidant (TRO) in the solution. This solution is then used as standard oxidant for test purpose.

In the utility drinking water for direct consumption by human body or for preparing beverages, hypochlorite is the typical residual oxidant in utility water and the typical TRO level is approximately 0.2 ppm. Hypochlorite is also one of the most common ROS produced in human body as well as external ROS ingested by human body. It is therefore appropriate to use hypochlorite as standard oxidant for its relevance to human body ROS, yet representative for the daily consumed beverages as well as the external ROS to human body. It is also confirmed by scientific research that hypochlorite is one of the ROS relevant to human body, e.g. hypochlorous acid (HOCl) is a precursor of free radicals in living systems (O. M. Panasenko et al. Biochemistry (Moscow), 2013, Vol. 78, No. 13, pp. 1466-1489); HOCl is one of the ROS in periodontal disease (Chong-Hou Sam et al. Journal of Dental Sciences, 2009, Vol. 4, Issue 2, pp. 45-54); and HOCl as an ROS participates in endothelial nitric oxide production (E A Jaimes et al. Hypertension, 2001, Vol. 38, Issue 4, pp. 877-883).

Considering the convenience and relevance to human body and beverages consumed, this TRO of 0.2 ppm concentration in water is therefore selected as the standard oxidant for the TAPEC method of the invention. This 0.2 ppm TRO solution can be easily prepared by adding hypochlorite in pure water to obtain a TRO concentration of 0.2 ppm.

This standard oxidant's ORP mV reading when measured by using the Ag/AgCl reference ORP meter is +550 mV.

Standard Antioxidant

Ascorbic acid is well recognized as an antioxidant to human body by medical practitioners, biochemists and nutritionists. Ascorbic acid solution is therefore chosen as the standard antioxidant for reference and titration ratio comparison. The standard antioxidant solution can be prepared by dissolving 800 mg ascorbic acid in 100 mL of pure water.

This standard antioxidant's ORP mV reading when measured by using the Ag/AgCl reference ORP meter is +175 mV.

Titration Test Using the Standard Antioxidant

In TAPEC method, a reference titration value of the standard antioxidant is required for comparison with the test beverages. To obtain the reference titration value, the standard antioxidant ascorbic acid solution is titrated into the standard oxidant 0.2 ppm hypochlorite solution of a pre-determined volume until the neutral ORP reference set point is reached.

When standard antioxidant ascorbic acid solution is progressively added to one litre of standard oxidant solution, the ORP reading of the standard oxidant solution will decrease progressively from +550 mV to smaller mV readings. Once the ORP in the titrated standard oxidant solution reached +375 mV (which is the set point of neutral solution ORP), the quantity “X” mL of the standard antioxidant ascorbic acid solution added is used as the reference titration volume for comparison with other test beverages.

In the standard antioxidant titration test, this “X” mL reference titration volume is 0.4 mL of standard antioxidant ascorbic solution. This 0.4 mL volume of titrated standard ascorbic acid antioxidant solution is assigned with a dimensionless unit—antioxidant scalar value (asv) of 1000 asv in the TAPEC method of the invention as the indicative antioxidant value.

Determination of TAPEC of Test Beverages

In TAPEC method, the antioxidant capacity of a test beverage is determined by titration test, and then a TAPEC antioxidant scalar value is assigned for the tested beverage to characterize its antioxidant capacity.

Similar as above, to determine the TAPEC antioxidant scalar value for a test beverage, the test beverage is titrated into the standard oxidant of a pre-determined volume till it reaches the complete neutralization ORP set point, i.e. +375 mV. The sample titration volume of beverage is then compared with the reference titration volume of standard antioxidant ascorbic acid solution to derive its antioxidant scalar value. As stated above, the reference titration volume of standard antioxidant ascorbic acid solution is 0.4 mL, which is assigned with a TAPEC value of 1000 asv.

To determine the TAPEC value of the test beverage, the ratio of reference titration volume 0.4 mL to the sample titration volume of the test beverage×1000 is defined as the TAPEC value of the test beverage. For instance, if the sample titration volume of the test beverage is “V” ml, the TAPEC value of said test beverage is calculated according to the following formula:

TAPEC value (asv) of the test beverage=(0.4 mL/(“V” mL))×1000

More specifically, to determine the antioxidant scalar value for a test beverage, the test beverage is titrated progressively into 1000 mL of 0.2 ppm TRO standard oxidant solution of hypochlorite till ORP of the standard oxidant solution reaches +375 mV. The amount of test beverages is then recorded as “V” ml.

The TAPEC antioxidant scalar value of the test beverage=0.4/“V”×1000=“xyz” TAPEC value (asv).

For a test beverage “V”=40 mL, the corresponding TAPEC value of the test beverage=0.4/40×1000=10 asv

The following table shows the typical TAPEC values for some of the common drinks using the above test methods and computation.

TABLE 1
TAPEC test results for common beverages

| | Sample Titration | Volume ratio | |
|---|---|---|---|
| | Volume of the | of Standard | |
| | Test Beverage | Antioxidant | TAPEC |
| | to neutralize | Ascorbic | value (asv) |
| Type of Test | the Standard | acid vs | of the test |
| Beverage | Oxidant (mL) | Test Beverage | Beverage |
| Ascorbic acid | 0.4 | 1.00 | 1000 |
| 800 mg/100 mL | | | |
| Tea | 1.5 | 0.267 | 267 |
| Coffee (black) | 2.7 | 0.148 | 148 |
| Orange juice | 13.5 | 0.030 | 30 |
| Whisky | 37.6 | 0.011 | 11 |
| Red wine | 1.94 | 0.206 | 206 |
| DC electrolysis | 0 | 0.0 | 0 |
| generated | | | |
| alkaline water | | | |

Stepwise TAPEC Test Procedure of the Invention

In the following, the test procedure of the method for determining the antioxidant capacity of a liquid sample will be described in a detailed stepwise manner. It is understood that not all the steps described in the following are indispensable for carrying out the method of the invention, and the method can be performed with certain steps omitted in actual practice.

Step 1—Calibration of pH and ORP Meters

pH and ORP meters are the two most important meters used in the whole TAPEC test. Both meters shall be calibrated by their respective standard reference liquids before use.

Step 2—Preparation of Pure Water

Pure water is used for three purposes in the TAPEC method:
b) As standard reference of a “neutral liquid”;
c) For preparation of standard antioxidant solution; and
d) For preparation of standard oxidant solution.

Pure water may be prepared through conventional procedure, such as by thermal distillation, vacuum flash distillation, ultra-filtration, or reverse osmosis process. However, several parameters of the pure water must be met before using in the method of present invention.
a) The electrical conductivity of the pure water must be <5 μs/cm indicating the dissolved content in the water is negligible for practical purpose.
b) pH must be near neutral range, i.e. pH at about 7.
c) Temperature of the pure water shall be at room temperature and consistent with the test samples.

After pure water is prepared, it is important to verify the ORP value of the pure water (neutral liquid) is at the neutral reference potential of +375 mV. Particularly, the ORP reading of the neutral pure water as prepared in step 2 shall be close to +375 mV versus Ag/AgCl reference electrode at room temperature and pH range close to 7. This is an important standard set point to determine the final neutralization end point during the titration of standard antioxidant and test beverage to standard oxidant.

Step 3—Preparation of Standard Oxidant

Into one liter (1 L) of pure water prepared in step 2, sodium hypochlorite solution is added till the TRO (Total Residual Oxidant) value of the solution reads 0.2 ppm so as to prepare the standard oxidant solution. TRO can be measured by colorimeter. If using 10% concentration solution of sodium hypochlorite, the volume of sodium hypochlorite solution added shall be 8 μL to 1000 mL pure water and the resulting TRO shall be 0.2 ppm. Standard oxidant ORP mV reading shall be +550 mV vs Ag/AgCl reference cell and pH shall be between 6.7 to 7 at room temperature.

Step 4—Preparation of Standard Antioxidant

Standard antioxidant solution is prepared by adding 800 mg of lab grade pure ascorbic acid into 100 mL of pure water prepared in step 2, so as to obtain a solution of ascorbic acid at a concentration of 800 mg/100 mL. The ORP reading of the resulting standard oxidant shall be +175 mV vs Ag/AgCl reference cell, pH 2.29.

Step 5—Preparation of Test Sample of Beverage to be Tested

TAPEC is intended for determining the antioxidant content in liquid beverages only, not for solid food or extraction thereof. Since antioxidant contents of beverages are already dissolved in the beverages hence no further extraction process is required. However, if there are undissolved solids or precipitates remain in the beverage, such as fruit pulp in fruit juice, grounded coffee powder etc., the beverage should be filtered in order to remove the undissolved contents to ensure no further changes from dissolving solid content during the test.

Subsequently, alkaline Lewis base content in the test beverage must be removed before the TAPEC test. The purpose of TAPEC value is to determine only the antioxidant that is able to neutralize human body free radical/ROS. The antioxidant content so determined must therefore be the free radical/ROS neutralizable antioxidant. Although Lewis-base alkaline are antioxidants from chemistry point of view, they donate paired electrons instead of individual free electron. Pair electron can form covalent bond but cannot donate free electron. Typical Lewis-base alkaline is hydroxide ions (OH⁻) in alkaline water which is useful in neutralizing the acidic H⁺ ions, but they cannot donate free electron to neutralize free radicals/ROS. These non-free electron donating antioxidants shall therefore be excluded from beneficial antioxidant content determination as they are of no antioxidant use to human body.

Since ORP meter is unable to differentiate whether the antioxidizing potential so measured is contributed by Lewis base alkaline pair electrons or free electron, it is therefore necessary to remove the Lewis base alkaline hydroxide ions from the test beverage (if any) before using the ORP meter for measurement.

Nevertheless, all natural beverages such as tea, coffee, wine, beer, fruit juices, hard liquor, natural water are all acidic or near to neutral. Therefore, by checking the pH of the test beverage, it will show whether the beverage contains Lewis base alkaline (particularly OH⁻).

If the beverage or water to be tested has a pH >7 or higher than the pure water used in the test, pH of the test sample shall be adjusted by adding hydrochloric acid (HCl) solution to neutralize the test sample close to pH 7 or pure water pH before the titration testing.

Step 6—Determination of the Reference Titration Volume of Standard Antioxidant to Standard Oxidant

To calibrate and calculate the antioxidant content TAPEC value, it is necessary to set a standard value as reference calibration scale/value. This is obtained by using the standard antioxidant to titrate the standard oxidant till it reaches the neutral state which is equal to the pure water ORP mV value.

Standard antioxidant solution of ascorbic acid is titrated into 1000 mL standard oxidant of 0.2 ppm TRO using a very small volume titration progression. The volume of standard antioxidant solution added is recorded and ORP reading of the mixed solution of each titration is checked till the reading reaches the stabilized state after each titration. The titration ends when the stabilized ORP reading reaches the pure water neutral ORP reading of +375 mV.

When using the standard antioxidant of ascorbic acid with concentration of 800 mg/100 mL to titrate the standard oxidant, the total titrated volume of the antioxidant added to bring the standard oxidant ORP from +550 mV to neutral state pure water ORP of +375 mV is 0.4 mL.

This 0.4 mL of standard antioxidant solution added is assigned with a TAPEC value of 1000 asv.

That means if any beverage can neutralize the 1000 mL standard oxidant with 0.4 mL titration volume, the TAPEC antioxidant value of that beverage is 1000, and it has the antioxidant free radicals/ROS neutralizing effect equivalent to ascorbic acid antioxidant solution of 800 mg/100 mL concentration.

For improving efficiency of the test procedure, in certain cases, this volume of 0.4 mL can be used as the reference titration volume in the calculation of antioxidant content value of a test sample without the actual titration test of the standard antioxidant.

Step 7—Determination of TAPEC Value for Test Sample

After determination of the reference titration volume of standard antioxidant as described in step 6, TAPEC value for other beverages can be determined by the following procedure.

After the pH correction of alkaline beverage in step 5 if required, test sample of beverage will be used to titrate the 1000 mL standard oxidant of ORP +550 mV with the same procedure as described in step 6. The titration ends when ORP of the standard oxidant and beverage mixture solution reaches the pure water neutral ORP reading of +375 mV. The neutral ORP reading indicates that all free radicals/ROS in the 1000 mL standard oxidant are fully neutralized.

If the volume of the beverage in neutralizing the standard oxidant is “V” mL, the TAPEC value of the beverage is calculated according to the following

TAPEC value (asv) of the test beverage=0.4/“V”×1000

If V=40 mL for the test beverage, the corresponding TAPEC value=0.4/40×1000=10 asv

If there are multiple beverages to be tested, the same procedure of step 7 can be repeated for other beverages to obtain their respective TAPEC antioxidant scalar value.

Factors Influencing TAPEC Value of Beverages

Due to the varieties of content in different types of beverages such as tea, coffee, orange juice, wine, liquor etc., their TAPEC values obtained by the TAPEC method of the invention may vary greatly, even for different test samples of same type of beverage. The variation in TAPEC value may be affected by the following aspects:

Preparation methods—Such as quantity and types of raw ingredients added, time of brewing, brewing temperature, grounded powder size, pasteurization, fermentation etc. Particularly, in view that Vitamin C is very sensitive to heat, pasteurization of orange juice or vitamin C containing drinks will see their TAPEC value varies in folds between pasteurized and non-pasteurized beverages.

Raw ingredients—Raw ingredients for same type of beverages will have great variation in their TAPEC antioxidant too. Good examples are different types of teas, coffee, wine, including different species of tea, types of grapes, of different country of origins, different batches of harvest etc., all will have different TAPEC values.

In view of the variation in TAPEC value, the present invention provides an efficient and convenient method for determining antioxidant capacity of liquid beverages, thus the antioxidant effect between different beverages can be easily compared, as well as that the ingested antioxidant contents can be easily monitored for a healthy diet.

Advantages of TAPEC of the Invention Over Prior Art ORAC

According to the above description regarding the TAPEC method for determining antioxidant capacity of liquid beverage, it can be seen that the TAPEC method achieves certain advantages and improvements over prior art method such as ORAC in the evaluation of antioxidant value of beverages. As an example, a comparison between aspects of TAPEC of the invention and prior art ORAC is summarized in following Table 2.

TABLE 2
Comparison chart between ORAC and TAPEC

| | | TAPEC |
|---|---|---|
| | ORAC | (Total Antioxidant |
| | (Oxygen Radical | Potential & |
| | Absorbance Capacity) | Energy Capacity) |
| Application | Solid Foods & Beverages | Beverages only |
| Standard | AAPH | Hypochlorite |
| Oxidant | | |
| (ROS Agent) | | |
| Biological | Reactive with water and | HOCl reacts with the |
| Relevance of | lipid to produce peroxyl | major classes of |
| ROS Agent | ROO• free radical | biologically important |
| | | molecules to form free |
| | | radicals |
| Standard | Trolox ® | Ascorbic Acid |
| Antioxidant | | |
| Neutralization | Fluorescent probe | ORP meter & pH meter |
| & Detection | (ORACFL) COBAS | Titration device |
| Instrument | FARA II analyzer | |
| Unit Reference | $\begin{matrix}Trolox Equivalent \\ ( TE ) over considered \\ concentration range = \\ \frac{\begin{matrix}slope of \\ regression curve \\ [ sample ]\end{matrix}}{\begin{matrix}slope of \\ regression curve \\ [ Trolox ]\end{matrix}}\end{matrix}$ | $\begin{matrix}TAPEC value ( asv ) = \\ \frac{\begin{matrix}reference titration \\ volume \times 1000\end{matrix}}{sample titration} \\ volume\end{matrix}$ |

As can be seen, although the TAPEC method of the invention is only intended for the evaluation of liquid samples (beverages), the present invention has improved over prior art in the selection of common chemical substances as standard oxidant and antioxidant, in the utilization of simple test devices, and in the assignment of an easily understandable and computable scale for comparison. With these advantages, the invention has developed an efficient and convenient method for determining antioxidant capacity of liquid beverages, which could promote healthy diet by improving awareness of antioxidant effects between different beverages through easier comparison.

In summary, disclosed herein is the Total Antioxidant Potential & Energy Capacity (TAPEC) method to determine the antioxidant capacity in beverages and test procedures thereof. The invention further provides test apparatus for performing the TAPEC method of the invention, and test kit comprising test apparatus and standard chemicals of the invention.

It is also understandable that the major inventive concept resides in the using of oxidation reduction potential as reference for determining oxidizing and antioxidizing state of solutions, in the utilization of simple test devices such as ORP meter and titration devices, in the selection of common oxidant (for example, hypochlorite) and antioxidant (for example, ascorbic acid), and in the assignment of an easily understandable scale (for example, antioxidant scalar value) for comparison, but the present invention is not limited to the above described embodiments.

While the embodiments described herein are intended as exemplary method and apparatus, it will be appreciated by those skilled in the art that the present invention is not limited to the embodiments illustrated. It is contemplated that the specific test meters, chemicals and scales can be replaced or substituted by other conventional means in the art, and the modified method and apparatus are still within the scope of the present invention as long as they can achieve the effects of the present invention.

What is claimed is:

1. A method for determining the antioxidant capacity of a liquid sample, comprising:
preparing the liquid sample by removing all undissolved contents therein;
measuring pH of the liquid sample and adjusting pH of the liquid sample to about 7 if the measured pH is higher than 7;
measuring oxidation reduction potential (ORP) of a standard oxidant;
determining a sample titration volume of the liquid sample necessary for bringing ORP of the standard oxidant of a pre-determined volume to a neutral reference value; and
calculating the antioxidant capacity based on the sample titration volume of the liquid sample.

2. The method according to claim 1, wherein
the standard oxidant is a solution of sodium hypochlorite.

3. The method according to claim 2, wherein
the standard oxidant has a total residual oxidant (TRO) value of 0.2 ppm, and/or an ORP reading of +550 mV vs Ag/AgCl reference cell.

4. The method according to claim 2, further comprising:
preparing the standard oxidant by adding at a ratio of 8 μL of a 10% HOCl solution into 1 L of pure water.

5. The method according to claim 1, further comprising:
preparing a standard antioxidant, the standard antioxidant is a solution of ascorbic acid.

6. The method according to claim 5, wherein
the standard antioxidant comprises ascorbic acid at a concentration of 800 mg/100 mL, and/or has an ORP reading of +175 mV vs Ag/AgCl reference cell.

7. The method according to claim 5, wherein
the standard antioxidant is prepared by adding at a ratio of 800 mg of pure ascorbic acid into 100 mL of pure water.

8. The method according to claim 5, further comprising:
determining a reference titration volume of the standard antioxidant necessary for bringing ORP of the standard oxidant of the pre-determined volume to the neutral reference value; and
calculating the antioxidant capacity based on the sample titration volume and the reference titration volume.

9. The method according to claim 8, wherein
the antioxidant capacity is calculated according to the following formula:
$$antioxidant scaler value ( asv ) = \frac{refe r ence titration volume \times 1000}{sample titration volume} .$$

10. The method according to claim 1, wherein
the neutral reference value is an ORP reading of +375 mV vs Ag/AgCl reference cell.

11. The method according to claim 1, further comprising:
preparing pure water;
using the pure water as a neutral liquid having ORP of the neutral reference value; and
using the pure water for the preparation of standard oxidant and standard antioxidant.

12. The method according to claim 1, wherein
pH of the liquid sample with pH higher than 7 is adjusted by adding hydrochloric acid solution.

13. The method according to claim 1, further comprising:
calibrating pH meter and ORP meter before measuring pH and ORP, respectively.

14. A test apparatus for performing the method according to claim 1, comprising:
a pH meter for measuring pH of a liquid sample;
an ORP meter for measuring ORP of a standard oxidant; and
a titration device for determining titration volume of the liquid sample.

15. A test kit for performing the method according to claim 1, comprising:
the test apparatus according to claim 14;
pure water;
hydrochloric acid; and
standard chemicals of the standard oxidant and the standard antioxidant.
